(19) United States
(12) Patent Application Publication
(10) Pub. No.: US 2026/0174949 A1
(43) Pub. Date: Jun. 25, 2026

(54) METHOD AND SYSTEM FOR CONTROLLING A FLUID PRESSURE IN A FLUID SYSTEM

(71) Applicant: Grundfos Holding A/S, Bjerringbro (DK)

(72) Inventor: Sten LINNELL, Bjerringbro (DK)

(21) Appl. No.: 19/124,535

(22) PCT Filed: Oct. 18, 2023
(86) PCT No.: PCT/EP2023/079043

§ 371 Date: Apr. 25, 2025

(30) Foreign Application Priority Data
Oct. 26, 2022 (DK) PA 2022 70520

(51) Int. Cl. A61M 3/00; E21B 47/06; F04B 49/06; F04B 49/20; F04D 15/02; G05D 7/06; G05D 11/13; H04L 67/12; H04Q 9/00

(52) U.S. Cl. CPC A61M 3/005; E21B 47/06; F04B 49/06; F04B 49/065; F04B 49/20; F04D 15/0218; G05D 7/0635; G05D 11/132; H04L 67/12; H04Q 9/00; A61M 2205/3334; A61M 2205/3344; A61M 2205/3379

(57) ABSTRACT

Method of controlling a pump to control a fluid pressure in a fluid system comprising a distribution source, a supply grid and a return grid for respective recipients and the pump wherein the method comprises: receiving a local differential pressure between the supply grid and the return grid at a first location; receiving a remote differential pressure between the supply grid and a the return grid at a second location further displaced along the supply line downstream from the pump than the second location; controlling operation of the pump based on the received data and on a local pressure set point; responding to a detected failure to receive the primary remote sensor data at least by modifying, in particular increasing, the local pressure set point and by controlling operation of the pump based on at least the received local sensor data and the modified local pressure set point.

### TECHNICAL FIELD

The present invention relates to the control of the fluid pressure in a fluid system, in particular in a fluid-based energy distribution systems, e.g. in a district energy system, such as a district heating system or a district cooling distribution network.

### BACKGROUND

In a fluid system, a fluid is transported from at least one distribution source via a grid of conduits to a plurality of recipients. To this end, the fluid system comprises a grid of supply conduits forming supply paths for transporting fluid to respective recipients and an associated grid of return conduits forming return paths for returning fluid from the recipients to the distribution source. For the purpose of the present disclosure, the supply conduits will also be referred to as supply lines and the return conduits will be referred to as return lines. The grids of supply and return conduits will also collectively be referred to the grid. For example, in a fluid-based energy distribution system, water or another fluid is used as a medium to transport thermal energy between a distribution source and a plurality of recipients. It will be appreciated that a fluid system, in particular a fluid-based energy distribution system may include one or more distribution sources. Examples of fluid-based energy distribution systems include heating systems and cooling systems. In particular, examples of fluid-based energy distribution systems include district energy systems, such as district heating or cooling systems. In a district heating system, water is heated at a district heating plant and transported to a plurality of consumers via a grid of supply conduits and returned from the consumers to the district heating plant via a grid of return conduits. District heating or cooling systems, which are sometimes also referred to as heat or cooling networks or grid heating or cooling systems, may have a variety of different sizes and, depending on their size, district heating systems may sometimes also be referred to as “campus heating system,” “Fernwärmesystem”, “Nahwärmesystem”, or the like. For the purpose of the present disclosure, the term district heating system is intended to refer to a heating system configured to distribute heat from one or more distribution sources to a plurality of different buildings via a network of supply and return lines, wherein the district heating system uses a fluid as a medium for transporting the heat through the network. Similarly the term district cooling system is intended to refer to a cooling system configured to distribute cooling from one or more distribution sources to a plurality of different buildings via a network of supply and return lines, wherein the district cooling system uses a fluid as a medium. Here and in the following, district heating and district cooling systems will collectively be referred to as district energy systems. It will be appreciated that some embodiments of a district energy system may be configured to provide heating as well as cooling.

Fluid-based energy distribution systems as disclosed herein also find applications as heating and/or cooling systems for individual buildings or other commercial or residential structures. Accordingly, the term fluid-based energy distribution system as used herein is intended to include district energy systems as well as heating and/or cooling systems of individual buildings or other commercial structures where thermal energy is distributed from one or more distribution sources to a plurality of recipients by transporting a fluid via a grid of supply and return lines.

The fluid system typically comprises at least one pump for pumping the fluid through a supply line of the system. The pump, in particular the pump speed, may be controlled by a control system. The control is typically based on a measured differential pressure between the supply and return lines. To this end, the system typically includes one or more pressure sensors for measuring the differential pressure between a supply line measurement point along a supply line of the fluid system and a corresponding return line measurement point along a return line of the fluid system.

It is generally desirable to provide a pressure control that is energy efficient and reliable. In this respect, on the one hand it is desirable to control the pump based on the measured differential pressure at a location remote from the pump, as the pressure remote from the pump more accurately reflects the differential pressure needed at the recipients, thus allowing for an energy-efficient control. On the other hand, installing pressure sensors remotely from the pump increases the risk that the communication between the control system and the pressure sensors fails.

In particular, there may be situations where the control system does not receive current pressure measurements from the pressure sensor, e.g. because of a failure of the sensor itself or because of a communication failure between the pressure sensor and the control system. The risk for a communication failure is particularly high when the communication between the pressure sensor and the control system is partly or completely based on wireless communications technology and/or when the pressure sensor is located far away from the control system.

Accordingly, it remains desirable to provide a reliable and energy-efficient control of the fluid pressure in the fluid system, which is robust against failures, such as sensor failures or communication failures.

### SUMMARY

In view of the foregoing, it remains desirable to provide a method and system for controlling fluid pressure in a fluid system, such as in a district energy system, that solve one or more of the above problems and/or that have other benefits, or that at least provide an alternative to existing solutions.

According to one aspect, disclosed herein are embodiments of a method of controlling operation of a pump to control a fluid pressure in a fluid system, such as a fluid-based energy distribution system. In particular, the fluid system comprises a supply grid of supply lines for transporting fluid from at least one distribution source to respective recipients, a return grid of return lines for returning fluid from the respective recipients to the distribution source, and a pump for pumping fluid through the fluid system, in particular through a supply line of the supply grid. Various embodiments of the method comprise:
receiving local sensor data indicative of a local differential pressure between a local supply line measurement location along a supply line of the supply grid and a local return line measurement location along a return line of the return grid;
receiving primary remote sensor data indicative of a primary remote differential pressure between a primary remote supply line measurement location along a supply line of the supply grid and a primary remote return line measurement location along a return line of the return grid, wherein the primary remote supply line measurement location is further displaced along the supply line downstream from the pump than the local supply line measurement location;
controlling operation of the pump based on at least the received local sensor data, the received primary remote sensor data and on a local pressure set point;
responding to a detected failure to receive the primary remote sensor data at least by modifying, in particular increasing, the local pressure set point and by controlling operation of the pump based on at least the received local sensor data and the modified local pressure set point.

In the presence of the primary remote sensor data, the process controls operation of the pump based not only on the local sensor data and on a local pressure set point, but additionally based on the received primary remote sensor data. As the primary remote sensor data is indicative of a differential pressure at a remote location, the pump control may take actual pressure measurements into account that are indicative of the actual pressure at a remote location within the fluid system, in particular at a location that, compared to the location of the pump, is close to the recipients, thereby allowing an improved and energy-efficient control of the pressure at the recipients.

Nevertheless, pump control based on sensor data may be continued despite a failure to receive the primary remote sensor data, thereby providing a reliable, uninterrupted pump control and allowing an energy efficient operation of the fluid system despite the occurrence of sensor or communication failures.

In some embodiments, the local pressure set point is indicative of a target differential pressure between the local supply line measurement location and the local return line measurement location. In other words, the pump control is, in the first place, based on a comparison of the local sensor signal with a local pressure set point, i.e. on a target value for the local sensor signal. The process may take the primary remote sensor signal into account by making the local pressure set point adaptive. In particular, during operation of the pump, the process may adapt the local pressure set point based on the primary remote sensor data, thus allowing the pump control to take the measured differential pressure at a remote location into account. In particular, the local pressure set point may be adapted based on a deviation of the primary remote sensor data from a primary remote pressure set point, in particular so as to reduce a magnitude of a deviation between the primary remote sensor data from the primary remote pressure set point. Accordingly, the primary remote pressure set point may be indicative of a target differential pressure between the primary remote supply line measurement location and the primary remote return line measurement location, i.e. indicative of a target differential pressure at a location that more accurately reflects the pressure needs of the recipients. Accordingly, the remote pressure set point may reflect what the control process ultimately intends to achieve, namely controlling the pressure in a vicinity of the recipients. Doing so by using a remote pressure set point to adaptively define a local pressure set point allows for a pump control that controls the pressure at a remote location while making the control process robust against a temporary loss of the remote sensor signal.

In particular, the pump control reacts to a detected failure to receive the primary remote sensor signal by modifying, in particular by increasing, the local pressure set point. This modification of the local pressure set point in a situation when no current primary remote sensor signal is received allows the control process to automatically account for the absence of current primary remote sensor data, thus avoiding the need for manual interference by an operator. In particular, in some embodiments, modifying the local pressure set point and controlling operation of the pump based on at least the received local sensor data and the modified local pressure set point comprises:
incrementally modifying, in particular incrementally increasing, the local pressure set point, and,
after each incremental modification of the local pressure set point, controlling operation of the pump based on at least the received local sensor data and the incrementally modified local pressure set point.

Accordingly, upon detection of a failure to receive the primary remote sensor data, the local pressure set point, on which the pump control is based in the first place, is gradually modified, in particular gradually increased. Accordingly, unnecessarily sudden changes in the control strategy, which might otherwise cause an unnecessarily high energy consumption, are avoided. In some embodiments, the process may select a rate of the incremental modification of the local pressure set point based on an estimated maximum change of the differential pressure at the recipients.

In some embodiments, the method comprises responding to a detected resumption of receipt of the primary remote sensor data at least by further modifying, in particular decreasing, the modified local pressure set point and by controlling operation of the pump based on at least the received local sensor data, the received primary remote sensor data and the further modified local pressure set point. Accordingly, upon resumption of receipt of the primary remote sensor data, the process may automatically return to a normal operational regime, without the need for any interference by an operator. Accordingly, a reliable and energy-efficient operation may be achieved. The further modification, of the local pressure set point may be performed gradually, in particular incrementally, as has been described above in connection with the modification of the local pressure sensor responsive to a detected failure. In particular, the further modification may include an adaptation of the local pressure set point based on an observed deviation of the primary remote sensor data from a primary remote pressure set point, as described below.

As mentioned above, the local pressure set point may be an adaptive set point, which is adapted based on the primary remote sensor data. In particular, in some embodiments, controlling operation of the pump based on at least the received local sensor data, the received primary remote sensor data and the local pressure set point comprises:
computing a deviation of the received primary remote sensor data from a primary remote pressure set point;
adapting the local pressure set point based on the computed deviation, in particular so as to reduce a magnitude (such as an absolute value) of the computed deviation.

Accordingly, during normal operation, in particular in the absence of a detected failure, the process performs a control of the differential pressure at the local measurement locations. This control is based on an adaptive local pressure set point, where the adaptation of the local pressure set point is based on the received primary remote sensor data. In particular, the adaptation of the local pressure set point may be based on an observed deviation of the received primary remote sensor data from a corresponding remote pressure set point. The process may thus adapt the local pressure set point used for the pump control so as to align the differential pressure at a primary remote location to a corresponding primary remote pressure set point. Accordingly, a desired pressure at a primary remote location of the fluid system, in particular at the recipients, may be achieved without the need for unnecessarily large safety margins in the local pressure set point, which would otherwise likely result in unnecessary energy consumption. In various embodiments of the method described herein, the local pressure control adapts to the actual needs of the fluid system at the recipients based on actual measurements, while providing a robust and energy-efficient fallback strategy in case of failure to receive the remote sensor data.

In some embodiments, the method comprises receiving auxiliary remote sensor data indicative of an auxiliary remote differential pressure between an auxiliary remote supply line measurement location along a supply line of the supply grid and an auxiliary remote return line measurement location along a return line of the return grid. Accordingly, modifying the local pressure set point and controlling operation of the pump based on at least the received local sensor data and the modified local pressure set point may comprise modifying the local pressure set point based on the auxiliary remote sensor data, received prior to the detected failure, and controlling operation of the pump based on at least the received local sensor data, the currently received auxiliary remote sensor data and on the modified local pressure set point. Accordingly, the process receives additional, auxiliary remote sensor data and utilizes the auxiliary remote sensor data to achieve a controlled reaction to a failure to receive the primary remote sensor data. The auxiliary remote supply line measurement location may be further displaced along the supply line downstream from the pump than the local supply line measurement location.

In particular, in some embodiments, modifying the local pressure set point based on the auxiliary remote sensor data comprises:
computing a maximum auxiliary remote differential pressure observed during a time window preceding the detected failure, and
modifying the local pressure set point based on a deviation of the currently observed auxiliary remote sensor data from the computed maximum auxiliary remote differential pressure.

Accordingly, when receipt of the primary remote sensor data fails, the process uses the auxiliary remote sensor data as a basis for the pump control instead. In doing so, the process accounts for the differential pressure at the auxiliary remote measurement locations as observed during the previous control preceding the detected failure, i.e. while the control was still based on the primary remote sensor data. Accordingly, upon failure to receive the primary remote sensor data, the process modifies the local pressure set point to account for the previously observed behavior of the auxiliary remote differential pressure, thus providing a reliable control while avoiding unnecessary energy consumptions due to excessive safety margins.

In some embodiments, modifying the local pressure set point based on the auxiliary remote sensor data further comprises:
computing a maximum rate of change of the auxiliary remote differential pressure observed during a time window preceding the detected failure, and
gradually modifying the local pressure set point at a rate corresponding to, in particular no larger than, the computed maximum rate of change.

Accordingly, the rate of change of the modification of the local pressure point in the event of a communication failure is based on the observed maximum changes of the remote auxiliary differential pressure, thus avoiding unnecessarily abrupt changes in the pressure control while avoiding too slow responses.

In some embodiments, the method comprises selecting a length of the time window responsive to a detected duration of the failure to receive the primary remote sensor data. In particular, initially, when the failure to receive the primary sensor data has only lasted a short period of time, the process may base the modification of the local pressure set point on the observed maximum auxiliary remote differential pressure and/or maximum observed change in auxiliary remote differential pressure over a time window of predetermined duration. When the communication failure lasts longer, the process may determine the observed maximum auxiliary remote differential pressure and/or the observed maximum change in auxiliary remote differential pressure over a correspondingly longer time period, thereby continuously adapting the modifications made to the local pressure set point to the estimated needs while avoiding unnecessarily strong end energy-inefficient changes. Generally, the duration of the time window may be selected based on an observed time scale of changes in differential pressure at the respective remote measurement locations. For example, in a district energy system, a suitable choice of time window may be between 10 min, and 1 month, such as between 30 min, and 1 week, such as between 30 min, and 24 h, such as between 1 h and 24 h.

In some embodiments, the method further comprises:
receiving remote sensor data indicative of respective remote differential pressures at respective sets of remote measurement locations, each set of remote measurement locations having a respective remote pressure set point associated with it,
selecting remote sensor data associated with a first one of the respective sets of respective remote measurement locations as the primary remote sensor data, and
selecting sensor data from associated with a second one of the respective sets of respective remote measurement locations as the auxiliary remote sensor data.

Accordingly, the process may receive remote sensor data from a plurality of different sets of remote measurement locations, i.e. from a plurality of remote pressure sensors, and automatically detect, in particular based on the received remote sensor data, which of the sets of remote measurement locations, i.e. which of the remote pressure sensors, to utilize as a source for the primary remote sensor data for the purpose of the pump control and which to utilize as a source for the auxiliary remote sensor data for use during a failure to receive the primary remote sensor data. Thereby, a further improved pump control is facilitated, as the process may base the control on measurements from the most relevant remote measurement location in the fluid system. Moreover, the selection of the most relevant remote measurement location may be based on actual measurements, thereby only requiring little, if any, a priori knowledge of the system behavior. Here, a set of remote measurement locations refers to a pair of a remote supply line measurement location and a corresponding remote return line measurement location between which a remote differential pressure is measured, by a suitable pressure sensor, e.g. by a differential pressure sensor or by a pair of individual pressure sensors. The sets of remote measurement locations may include respective supply line measurement locations that are each further displaced along the supply line downstream from the pump than the local supply line measurement location.

In some embodiments, selecting comprises:
for each of the sets of remote measurement locations, determining a deviation between the remote sensor data measured at said set of remote measurement locations and the remote pressure set point associated with said set of remote measurement locations, and
selecting the remote sensor data of the set of remote measurement locations having the smallest deviation as the primary remote sensor data.

Accordingly, the process selects the measurement location requiring the tightest pressure control as the source for the primary sensor data, thus providing reliable and energy efficient control.

In some embodiments, the method further comprises selecting the remote sensor data from the set of remote measurement locations having the second to smallest deviation as the auxiliary remote sensor data. Accordingly, in case of a failure to receive the primary remote sensor data, control can proceed based on the set of remote measurement locations requiring the second to tightest pressure control as the source for the remote sensor data, thus requiring reliable and energy efficient control even in the event of a communication failure. It will be appreciated that the process may establish a prioritized list of additional sets of remote measurement locations, such that remote sensor data from these may be used as lower-ranking auxiliary remote sensor data in an analogous fashion as further fallback data in case the process fails to receive the primary remote sensor data as well as the higher-ranking auxiliary remote sensor data.

It will be appreciated that the set of remote measurement locations having the smallest deviation may vary over time, in particular depending on variations in the load or in the load distribution within the fluid system. Similarly, the set of remote measurement locations having the second to smallest deviation and the order of the sets of remote measurement locations in the prioritized list may vary over time. Accordingly, in some embodiments the process may repeat, e.g. intermittently or at regular time intervals, the steps of determining the deviations and selecting the remote sensor data of the set of remote measurement locations having the smallest deviation as the primary remote sensor data. For example, in a district energy system, a remote measurement location in an area with primarily office buildings may more likely be selected as the source of the primary remote sensor data during office hours while an area with primarily residential buildings may more likely be selected as the source of the primary remote sensor data during non-office hours.

In some embodiments, the sets of remote measurement locations are located at respective peripheral portions of the supply and return grids, respectively, i.e. in a proximity of recipients. Accordingly, a pressure control is achieved that can take account of the observed remote pressure conditions at the recipients, while being robust against communication and/or sensor failures. In this respect, the supply and return grids may be considered to have their respective roots at the distribution source and the peripheral portions of the supply and return grids may be defined as the parts having the largest distances from the distribution source.

Generally, the supply grid may include one or more main supply lines from which one or more branch lines branch off, such that each branch line fluidly connects one or recipients with the main supply line. It will be appreciated, however, that the supply grid may have a different, including a more complicated, grid topology. It will be appreciated that distances along the supply line between two respective locations within the supply grid may be measured as a length of a supply path along the supply lines of the supply grid between these locations. If there are more than one path, the distance may be determined as the shortest path. Similarly, distances along the return line between two respective locations within the return grid may be measured as a length of a return path along the return lines of the return grid between these locations.

The local supply line measurement location and the local return line measurement location are preferably located at or near the pump, e.g. at or near a distribution source. The primary remote supply line measurement location is preferably further away along the supply line from the pump than the local supply line measurement location. The distance between a supply line measurement location and the pump along the supply line may be defined as the length of the supply line between the supply line measurement location and the point along the supply line that is closest to the pump. When the pump is operationally coupled to the supply line, the point along the supply line closest to the pump corresponds to the point where the pump is operationally coupled to the supply line. Similarly, the primary remote return line measurement location is preferably further away along the return line from the pump than the local return line measurement location. When the pump is operationally coupled to the supply line, the point along the return line closest to the pump may be considered as being the point along the return line that has the shortest distance in space to the pump. Yet similarly, the auxiliary remote supply line measurement location and the auxiliary remote return line measurement location are further away along the supply line or the return line, respectively, from the pump than the corresponding local supply line and return line measurement locations, respectively.

In some embodiments, detecting a failure to receive the primary remote sensor data comprises detecting a failure to receive the primary remote sensor data for at least a predetermined minimum outage period, thus avoiding unnecessary reactions to short-term outages. The predetermined minimum outage period may be selected based on an observed time scale of changes in differential pressure at the respective remote measurement locations. For example, in a district energy system, a suitable choice of a predetermined minimum outage period may be between 5 min, and 1 h, such as between 10 min, and 30 min. e.g. about 15 min.

Generally, the fluid may be water or another liquid. The distribution source may be an energy distribution source for providing heated or cooled fluid, such as water. In particular, the distribution source may be a boiler, a chiller bank, a district heating plant, a district cooling plant, or another apparatus for heating or cooling the fluid. The distribution source may also be a pump station, e.g. in case of an extended fluid system. It will be appreciated that a fluid system, in particular a fluid-based energy distribution system may include one or more distribution sources. In embodiments with more than one distribution source, the distant portions of the peripheral portions of the supply and return grids may be defined as the parts having the largest distances from the closest distribution source.

The pressor sensors for measuring differential pressure at the sets of local or remote measurement locations may be differential pressure sensors or pairs of pressure sensing devices that separately measure the individual pressures in the supply and return lines, respectively.

The present disclosure relates to different aspects including the method described above and in the following, corresponding apparatus, systems, methods, and/or products, each yielding one or more of the benefits and advantages described in connection with one or more of the other aspects, and each having one or more embodiments corresponding to the embodiments described in connection with one or more of the other aspects and/or disclosed in the appended claims.

In particular, according to another aspect, disclosed herein are embodiments of a control system for controlling operation of a pump to control a fluid pressure in a district energy system, the control system being configured to perform the steps of the method as disclosed herein. The control system may be a PLC-based system, a computer-implemented system, a SCADA system, or the like. The control system may control a single pump or multiple controllable components of the fluid system.

Embodiments of the methods disclosed herein may be computer-implemented. Accordingly, further disclosed herein are embodiments of a data processing system configured to perform the steps of one or more of the methods described herein. In particular, the data processing system may have stored thereon program code adapted to cause, when executed by the data processing system, the data processing system to perform the steps of one or more of the methods described herein.

Embodiments of the control system and/or of the data processing system may be embodied as a single computer or other data processing device, or as a distributed system including multiple computers and/or other data processing devices, e.g. a client-server system, a cloud-based system, etc. The control system and/or the data processing system may include a data storage device for storing the computer program and sensor data. The control system and/or the data processing system may include a communications interface for receiving sensor data from one or more pressure sensors. The control system and/or the data processing system may receive the sensor data from the one or more pressure sensors via a suitable wired or wireless communicative connection, e.g. via a suitable communications network.

The control system and/or the data processing system may provide a user-interface for allowing a user to monitor operation of the pump and/or other data associated with the operation of the fluid system. The data processing system may also issue warnings or alerts or other notifications, e.g. responsive to detected communication failures, e.g. audible or visual alerts, alerts communicated via e-mail, SMS, or other forms of notifications, and/or the like.

Another aspect disclosed herein relates to a fluid system. The fluid system comprises:
at least one distribution source for providing a fluid,
a supply grid of supply lines for feeding the fluid from the distribution source to a plurality of recipients,
a return grid of return lines for returning fluid from the plurality of recipients to the distribution source,
a pump for pumping fluid through a supply line of the supply grid;
a control system as described herein for controlling the pump,
at least one local pressure sensor communicatively coupled to the control system and configured for providing local sensor data indicative of a local differential pressure between a local supply line measurement location along a supply line of the supply grid and a local return line measurement location along a return line of the return grid,
at least one primary remote pressure sensor communicatively coupled to the control system and configured for providing primary remote sensor data indicative of a primary remote differential pressure between a primary remote supply line measurement location along a supply line of the supply grid and a primary remote return line measurement location along a return line of the return grid, wherein the primary remote supply line measurement location is further displaced along the supply line downstream from the pump than the local supply line measurement location.

Yet another aspect disclosed herein relates to embodiments of a computer program configured to cause a data processing system to perform the acts of the method described above and in the following. A computer program may comprise program code means adapted to cause a data processing system to perform the acts of the method disclosed above and in the following when the program code means are executed on the data processing system. The computer program may be stored on a computer-readable storage medium, in particular a non-transient storage medium, or embodied as a data signal. The non-transient storage medium may comprise any suitable circuitry or device for storing data, such as a RAM, a ROM, an EPROM, EEPROM, flash memory, magnetic or optical storage device, such as a CD ROM, a DVD, a hard disk, and/or the like.

### BRIEF DESCRIPTION OF THE DRAWINGS

Preferred embodiments will be described in more detail in connection with the appended drawings, where

FIG. 1 schematically shows an embodiment of a fluid system.

FIG. 2 schematically shows another embodiment of a fluid system.

FIG. 3 shows a number of schematic diagrams, each illustrating the differential pressure between the supply and return lines of a fluid system as a function of distance from the pump.

FIG. 4 schematically shows a more detailed view of a part of a fluid system.

FIG. 5 schematically shows a portion of another embodiment of a fluid system.

FIGS. 6A and 6B show state diagrams of two examples of a control process for operating a pump of a fluid system.

FIGS. 7A and 7B schematically illustrate a process for operating a pump of a fluid system.

FIGS. 8A-B illustrate an example of the pump operation in a fluid system with more than one remote pressure sensors.

FIGS. 9A-C illustrate the time-varying primary remote sensor data, the highest ranking auxiliary remote sensor data and the local sensor data in an embodiment of a fluid system.

FIGS. 10A-10C illustrate an example of the pump control in a situation where a failure to receive the primary remote sensor data occurs.

### DETAILED DESCRIPTION

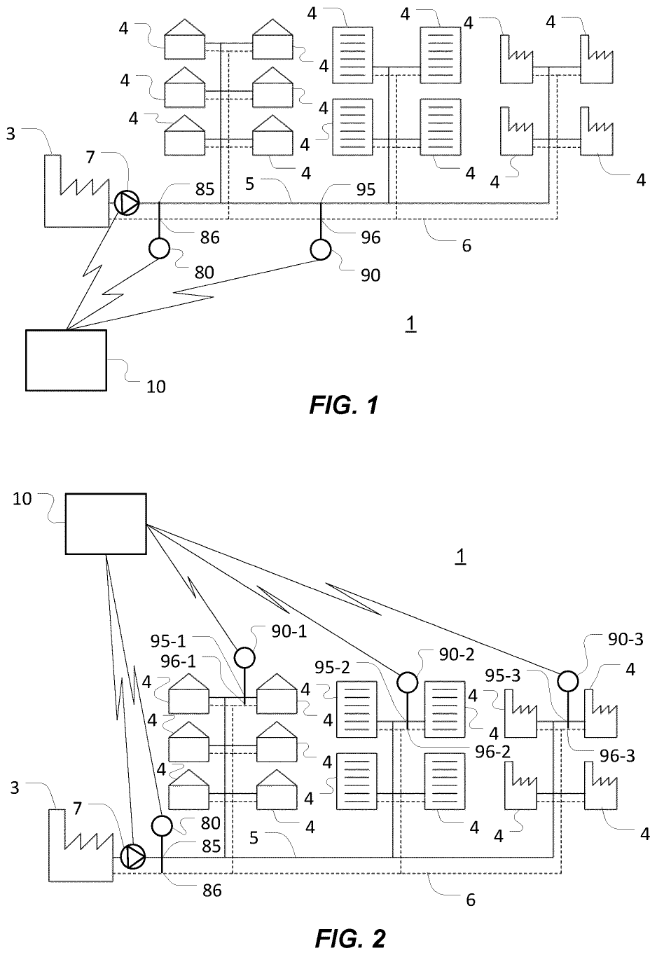

FIG. 1 schematically shows an embodiment of a fluid system, generally designated by reference numeral 1. The fluid system 1 comprises a distribution source 3 for providing a fluid. In the present example, the fluid system 1 is a district heating system and the distribution source 3 is a district heating plant which provides heated water. However, it will be appreciated that various embodiments of the methods, apparatus and system disclosed herein may also be applied to other types of fluid systems, in particular to other types of fluid-based heating or cooling systems.

The fluid system 1 comprises a supply grid of supply lines 5 for transporting the fluid from the distribution source 3 to a plurality of recipients 4. In the example of a district heating system, the recipients may be domestic or commercial buildings, such as family homes, apartment buildings, office buildings or other commercial buildings. It will be appreciated, however, that other embodiments may include other types of recipients. Moreover, different embodiments may have different numbers of recipients. As illustrated in FIG. 1, the supply grid of supply lines 5 may include a main supply line and a plurality of branch lines, branching off from the main supply line to respective recipients 4. However, other embodiments may have a different grid structure. The fluid system 1 further includes a return grid of return lines 6 for returning fluid from the respective recipients 4 to the distribution source 3. The supply lines and/or return lines may be pipes or other suitable types of conduits.

The fluid system 1 further comprises a pump 7 for pumping fluid between the distribution source 3 and the recipients 4. The pump may be a centrifugal pump or another suitable type of pump. The pump may be an electrical pump. The pump may be a pump whose pump speed can be controlled, e.g. by means of a frequency converter or by another suitable speed control circuit. In the example of FIG. 1, the pump 7 is shown separate from the distribution source 3, e.g. in the form of a separate pump station. However, in other embodiments, the pump may be an internal pump of the distribution source. The pump 7 may be operationally coupled to the supply line 5 and pump fluid through the supply line 5 toward the recipients 4. Alternatively or additionally, the fluid system may include a pump in the return line. Yet further, it will be appreciated that some embodiments of a fluid system may include more than one pump, some or all of which may be controlled by an embodiment of the control method as described herein, or otherwise.

The fluid system 1 further comprises a local differential pressure sensor 80 configured to measure the differential pressure between respective local measurement locations 85 and 86 along the supply line 5 and the return line 6. The measurement locations 85 and 86 are in the proximity of the pump 7. The local measurement location 85 along the supply line is located downstream from the pump 7 along the supply line 5. The local measurement location may be located immediately downstream from the pump 7, or even integrated into the pump 7, or it may be located displaced and downstream from the pump along the supply line, e.g. displaced for 1 m or more, such as 10 m or more or even further away from the pump 7. The local measurement location 86 is positioned at a suitable position along the return line, preferably in the vicinity of the local measurement location 85. Typically, the supply and return lines are arranged next to each other or otherwise not far removed from each other. It will further be appreciated that, instead of a differential pressure sensor 80, the system may comprise separate pressure sensors for measuring the fluid pressure at the local supply line measurement location 85 along the supply line 5 and at the local return line measurement location 86 along the return line 6, respectively, thus allowing a local differential pressure to be derived from the individual pressure readings. Generally, a differential pressure sensor may include capillary pipes connected to the respective measurement locations and a sensor for measuring a pressure difference between the pipes. Alternatively, individual pressure sensors may be arranged at the measurement locations where the sensors are electrically or otherwise communicatively connected so as to determine a differential pressure between the measurement locations.

The fluid system 1 further comprises a remote differential pressure sensor 90 configured to measure the differential pressure between respective remote measurement locations 95 and 96 along the supply line 5 and the return line 6. The measurement locations 95 and 96 are remote from the pump 7, i.e. the remote supply line measurement location 95 is located downstream from the pump along the supply line 5, and further displaced along the supply line 5 from the pump 7 than the local supply line measurement location 85. The remote measurement location 96 is positioned at a suitable position along the return line, preferably in the vicinity of the remote measurement location 95. Accordingly, the remote return line measurement location 96 may be located upstream from the local return line measurement location 86 along the return line 6, and further displaced from the pump 7 than the local return line measurement location 86. It will further be appreciated that, instead of a remote differential pressure sensor 90, the system may comprise separate remote pressure sensors for measuring the fluid pressure at the remote supply line measurement location 95 along the supply line 5 and at the remote return line measurement location 96 along the return line 6, respectively, thus allowing a remote differential pressure to be derived from the individual pressure readings. In typical fluid systems, in particular in district heating systems, the distance between the pump and the remote pressure sensor 90 may be rather large, e.g. at least 1 km or several kilometers.

The fluid system 1 further comprises a control system 10 for controlling operation of the pump 7. The control system 10 may be a computer-implemented control system, a PLC-based system and/or the like. The control system 10 may implement a SCADA system or another suitable type of control function. In the example of FIG. 1, the control system is illustrated separate from the distribution source 3. However, it will be appreciated that the control system 10 may be implemented as an integral part of the distribution source 3.

The control system 10 is operationally coupled to the pump 7, e.g. by a wired or wireless connection and configured to adjust a pump speed of the pump 7 so as to control the local differential pressure provided by operation of the pump. To this end, the local differential pressure sensor 80 is communicatively coupled to the control system 10, e.g. by a wired or wireless connection, so as to allow the control system 10 to receive local sensor data indicative of the local differential pressure between the local measurement points 85 and 86.

Additionally, the control system 10 is further communicatively coupled to the remote differential pressure sensor 90, e.g. by a wired or wireless connection, so as to allow the control system 10 to receive remote sensor data indicative of the remote differential pressure between the remote measurement points 95 and 96.

The control system 10 is typically located at the distribution source 3. As the remote pressure sensor 90 may be positioned far away from the distribution source 3, wireless communication between the remote pressure sensor 90 and the control system 10 may be preferred so as to avoid the need for long wired data connections. For example, the remote pressure sensor 90 may communicate remote sensor data to the control system 10 via a suitable communications network, such as a cellular telecommunications network or via another suitable wireless interface to a communications network, such as the Internet.

It is desirable that the control system 10 is configured to adjust the pump speed of the pump 7 such that a suitable differential pressure is obtained at the recipients 4. When the pump 7 is controlled based on local pressure measurements by the local pressure sensor 80 alone, the control system 10 may adjust the pump speed such that the measured local differential pressure corresponds to a predetermined local pressure set point. However, the local control has the disadvantage that there may be a large difference between the local pressure at the location of the pump 7 and the resulting residual differential pressure at the recipients 4. Moreover, the difference between these pressures may depend on the current flow through the system, i.e. on the operational status of the recipients 4. Therefore, when adjusting the pump speed based on local pressure measurements from local pressure sensor 80 alone, an operator of the system may need to add substantial safety margin to the local pressure set point to which the local differential pressure is controlled, so as to ensure that the residual differential pressure at the recipients 4 does not decrease below the needed pressure at the recipients, even during high-flow conditions.

Therefore, in various embodiments disclosed herein, the control system 10 further bases the pump control on remote sensor data obtained from one or more remote pressure sensors. Specifically, in the example of FIG. 1, the control system 10 receives remote sensor data from remote differential pressure sensor 90, which is placed at a central location of the supply and return grids, remote from the pump 7.

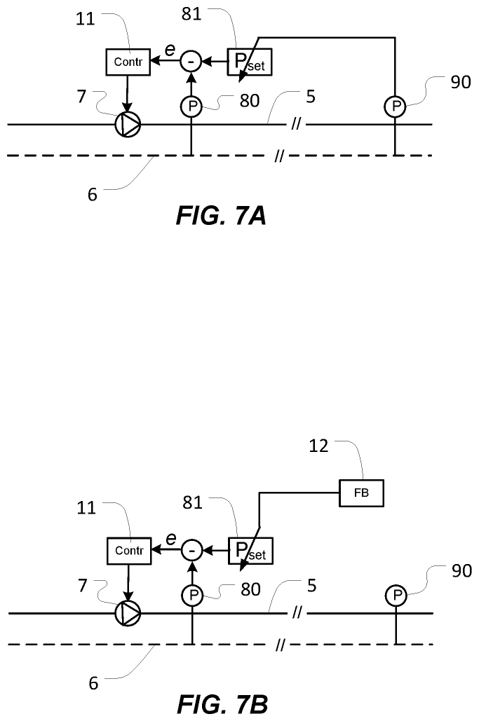

An example of a control process for controlling the pump 7 based on the local sensor data and on the remote sensor data will be described in more detail below with reference to FIGS. 6A and 7A-B. In particular, the described control process also accounts for situations where the communication between the control system 10 and the remote pressure sensor 90 fails, e.g. due to an outage of the wireless communication, due to damaged wired data connections, due to a sensor failure or due to other types of failures.

FIG. 2 schematically shows another embodiment of a fluid system 1. The fluid system 1 of FIG. 2 is similar to the system of FIG. 1 in that it comprises a distribution source 3, a supply grid of supply lines 5, a return grid of return lines 6, a plurality of recipients 4, a pump 7, a control system 10 and a local differential pressure sensor 80, all as described in connection with FIG. 1.

The fluid system 1 of FIG. 2 differs from the system of FIG. 1 in that the fluid system 1 of FIG. 2 includes more than one remote pressure sensors 90-1 through 90-3. The remote pressure sensors 90-1 through 90-3 are positioned in peripheral portions of the supply and return grids, i.e. far remote from the distribution source 3 and the pump 7 and close to respective recipients 4. Each of the remote pressure sensors measures a differential pressure between a respective remote supply line measurement locations 95-1 through 95-3 along the supply line 5 and corresponding remote return line measurement locations 96-1 through 96-3, respectively, along the return line 6. Each of the remote pressure sensors 90-1 through 90-3 is communicatively coupled to the control system 10, e.g. via wireless or wired communication as was described in connection with the remote pressure sensor 90 of FIG. 1. As was described in connection with FIG. 1, instead of differential pressure sensors, the system may comprise multiple pairs separate pressure sensors in the supply and return lines, respectively, from which respective differential pressures may be derived. Moreover, while there are three remote pressure sensors shown in FIG. 2, it will be appreciated that other embodiments may include a different number of remote pressure sensors.

Accordingly, in the embodiment of FIG. 2, the control system 10 bases the pump control on local sensor data from the local pressure sensor 80 and, additionally, on remote sensor data from one or more of the remote differential pressure sensors 90-1 through 90-3. In particular, as will be described in greater detail below, the control system 10 may select a primary remote pressure sensor among the plurality of pressure sensors 90-1 through 90-3 and base the pump control on the local sensor data from the local pressure sensor 80 and, additionally, on primary remote sensor data from the selected primary remote pressure sensor.

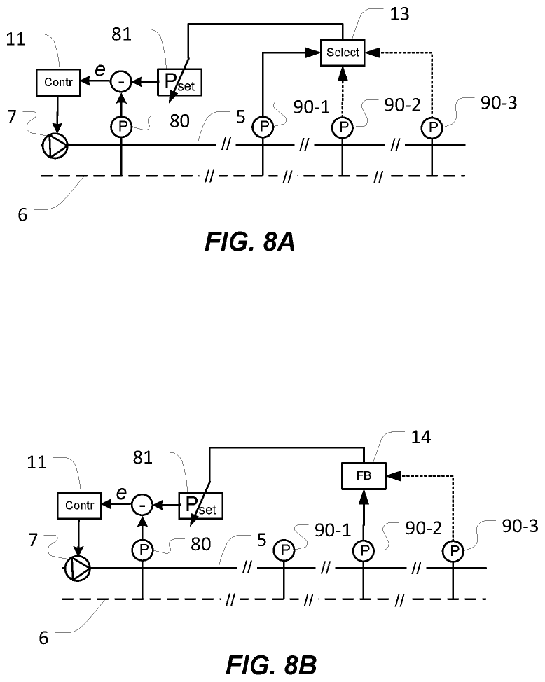

In case of failure to receive the primary remote sensor data from the selected primary remote pressure sensor, the control system 10 may temporarily base the pump control on the local sensor data from the local pressure sensor 80 and, additionally, on auxiliary remote sensor data from an auxiliary remote pressure sensor selected from the plurality of remote pressure sensor 90-1 and 90-3, different from the primary remote pressure sensor. To this end, the control system 10 may further select an auxiliary remote pressure sensor from the plurality of remote pressure sensors 90-1 through 90-3, different from the selected primary remote pressure sensor. An example of such a control process will be described in greater detail below with reference to FIGS. 6B and 8A-B.

Remote sensor data indicative of the differential pressure at the periphery or edge of the supply and return grids more accurately reflects the actual differential pressure at the recipients 4, thus allowing for a more energy-efficient pump control while ensuring a sufficiently high differential pressure at the recipients during various flow conditions.

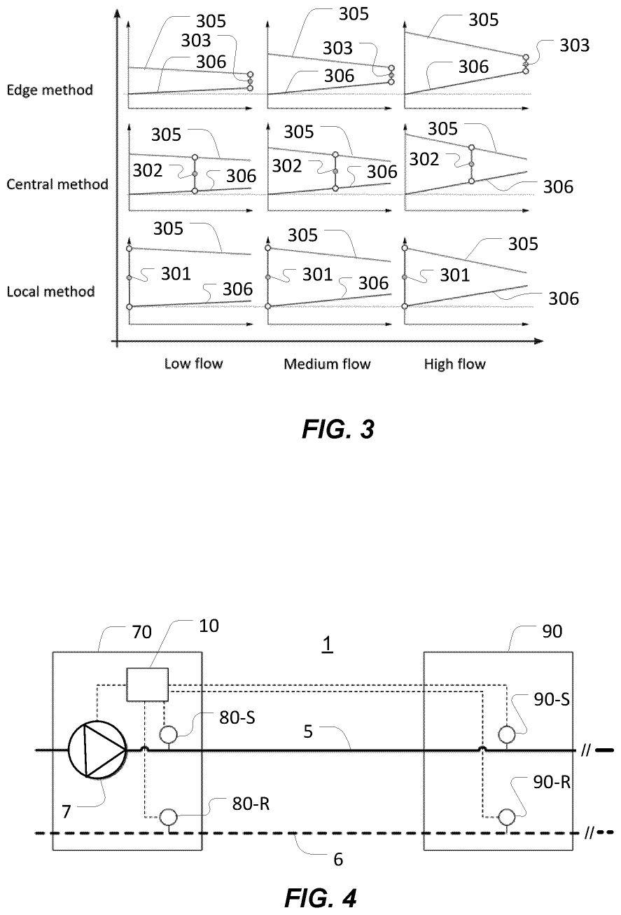

This is schematically illustrated in FIG. 3. In particular, FIG. 3 shows a number of schematic diagrams, each illustrating the pressure 305 along the supply and the pressure 306 along the return line of a fluid system as a function of distance from the pump. The gap between the lines 305 and 306 thus represents the differential pressure as a function of distance from the pump.

In FIG. 3, the diagrams are arranged in three rows and three columns. Each column of diagrams shows the differential pressure for a certain flow condition, where different columns correspond to different flow rates through the system. The diagrams of the left column show a situation at low flow conditions, the diagrams of the center column show a situation at medium flow and the right column of diagrams shows the situation at high flow.

The rows correspond to different placements of the pressure sensors and, hence, to different control paradigms. The bottommost row of diagrams illustrates the differential pressure in a system where the pump control is entirely based on a local pressure measurement. To ensure that the residual pressure at the recipients, furthest away from the pump, is sufficiently high, the local differential pressure 301 has to be controlled compared to a local pressure set point large enough to ensure a sufficiently high residual pressure at the recipients even during high-flow conditions. However, this results in an unnecessarily high residual pressure during medium and, particularly, during low flow conditions, which in turn renders this control scheme energy-inefficient.

The middle row of graphs shows a control scheme based on a remote pressure sensor position in a central portion of the supply and return grids, e.g. as illustrated by remote pressure sensor 90 of the system of FIG. 1. The measured differential pressure 302 by the centrally placed remote pressure sensor more closely resembles the residual pressure at the recipients. Accordingly, when basing the pump control on the remote pressure data from the centrally placed pressure sensor, the pump control can be made more efficient.

A further improvement of the energy efficiency can be achieved when the pump control is based on pressure measurements by one or more remote pressure sensors at the edge of the supply and return grids, e.g. as illustrated by the remote pressure sensors 90-1 through 90-3 in the example of FIG. 2. Examples of the differential pressure resulting from a pump control using differential pressure measurements 303 at the edge of the supply and return grids under different flow conditions are shown in the uppermost row of graphs in FIG. 3. As will be appreciated from a comparison of the different control schemes, the edge-based approach provides a more energy efficient control of the pump under medium and low flow conditions, as the differential pressure at the pump may be kept smaller during low and medium flow conditions, while still being able to ensure sufficient differential pressure at the recipients.

However, the risk of communication failure increases when the number of sensors increases and when the sensors are distributed across a large geographic area covered by the supply and return grids. Accordingly, when basing the pump control on remote sensor data, a control process should preferably account for situations where the control system fails to receive the remote sensor data. An uninterrupted pump control, in particular an uninterrupted automatic pump control, should be ensured even in such situations. This problem is particularly pronounced in the context of the edge-based control scheme based on pressure sensors at the periphery of the grid.

Embodiments of a control process utilizing remote sensor data and taking account of possible failure situations will be described in greater detail below.

FIG. 4 schematically shows a more detailed view of a part of a fluid system 1. The fluid system 1 of FIG. 4 is similar to the system of FIG. 1 in that it comprises a supply grid of supply lines 5 for feeding fluid from a distribution source (nor explicitly shown in FIG. 4) to a plurality of recipients (nor explicitly shown in FIG. 4) and a return grid of return lines 6 for returning fluid from the recipients to the distribution source. In FIG. 4, a supply grid with only a single supply line and a return grid with only a single return line are shown for ease of illustration, but it will be appreciated that most systems will include more complicated supply and return grids. The fluid system 1 further comprises a pump station 70 comprising a pump 7, a control system 10, and a pair of local pressure sensors 80-S and 80-R for measuring the fluid pressure in the supply line 5 and the return line 6, respectively, at the location of the pump station 70. The local pressure sensors 80-S and 80-R may be integrated into the pump station 70, as schematically illustrated in FIG. 4, or they may be arranged separately from the pump station instead. In any event, the local pressure sensors 80-S and 80-R are communicatively coupled to the control system 10, such that the control system 10 receives local sensor data indicative of the measured local pressures in the supply line 5 and the return line 6, respectively, and, hence, indicative of the local differential pressure, at or near the location of the pump station 90. In particular, the control system 10 may derive the local differential pressure between the supply line 5 and the return line 6 at the location of the pump station 70 from the received local sensor data. It will be appreciated that the pump station 70 may include, or be connected to, a differential pressure sensor instead, which may directly provide the differential pressure. While the control system 10 is shown as integrated into the pump station, it will be appreciated that the control system may be implemented separately from the pump station, and communicatively coupled to the pump station, instead. The pump 7 is coupled to the supply line 5 and pumps fluid through the supply line 5 towards the recipients. However, in other embodiments, the pump may be provided operationally coupled to the return line. Yet further, some embodiments may include more than one pump.

The fluid system 1 further comprises a remote measurement station 90, which is located remote and spaced apart from the pump station 70, e.g. more than 10 m from the pump station, such as more than 100 m, such as more than 1 km away from the pump station. The remote measurement station 90 comprises remote pressure sensors 90-S and 90-R for measuring the fluid pressure in the supply line 5 and the return line 6, respectively, at the location of the remote measurement station 70. The remote pressure sensors 90-S and 90-R are communicatively coupled to the pump station 70 and, in particular, to the control system 10, such that the control system 10 receives remote sensor data indicative of the measured pressures in the supply line 5 and the return line 6, respectively, and, hence, indicative of the differential pressure, at the location of the remote measurement station 90. To this end, the control system 10 may derive the differential pressure between the supply line 5 and the return line 6 at the location of the remote measurement station 90 from the received remote sensor data. It will be appreciated that the measurement station may include a differential pressure sensor instead and directly provide the differential pressure.

The control system 10 thus receives local sensor data from local pressure sensors 80-S and 80-R as well as remote sensor data from the remote pressure sensors 90-S and 90-R. The control system 10 is configured to control operation of the pump 7 based on the thus received local and remote sensor data, e.g. as described below with reference to FIGS. 6A and 7A-B.

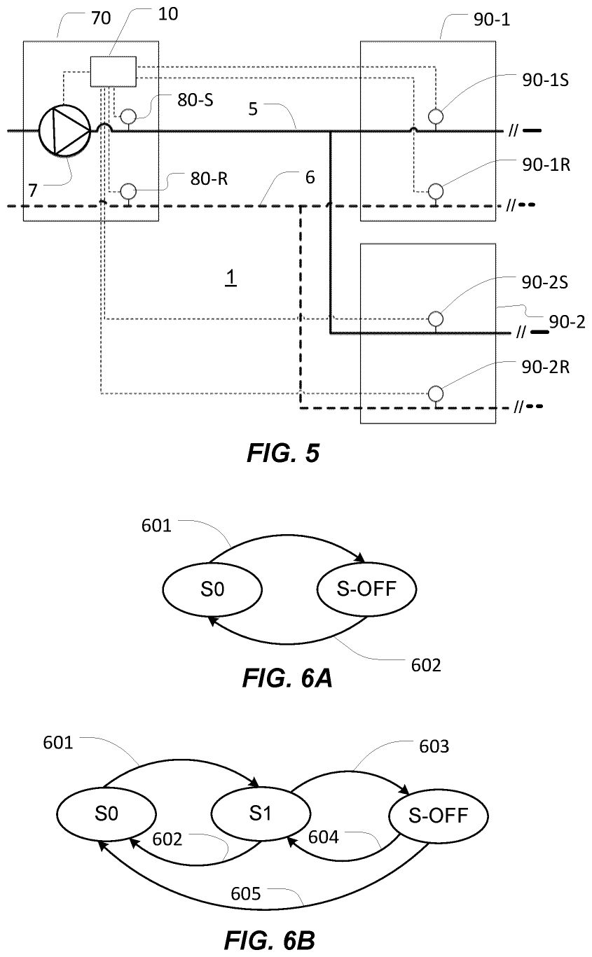

FIG. 5 schematically shows a portion of another embodiment of a fluid system 1. The fluid system 1 of FIG. 5 is similar to the system of FIG. 4 in that it comprises a pump station 70 with a pump 7, a control system 10 and local pressure sensors 80-S and 80R, all as described in connection with FIG. 4. The fluid system 1 further comprises a primary remote measurement station 90-1, which includes primary remote pressure sensors 90-1S and 90-1R, all as described in connection with remote measurement station 90 of the system of FIG. 4. The fluid system 1 of FIG. 5 differs from the system of FIG. 4 in that the fluid system 1 of FIG. 5 further comprises an additional, auxiliary remote measurement station 90-2. The auxiliary remote measurement station 90-2 is placed remote and spaced apart from the pump station 70 and remote and spaced apart from the primary remote measurement station 90-1, e.g. in a different part of a district energy grid, along a different branch supply line and corresponding branch return line than the primary remote measurement station 90-1, or the like. Like the primary remote measurement station 90-1, the auxiliary remote measurement station 90-2 includes a pair of pressure sensors, namely in this case auxiliary remote pressure sensors 90-2S and 90-2R for measuring fluid pressure in the supply line 5 and the return line 6, respectively, at the location of the auxiliary remote measurement station 90-2. The auxiliary remote pressure sensors 90-2S and 90-2R are communicatively coupled to the control system 10 and forward corresponding auxiliary remote sensor data to the control system 10, all as described with respect to the previously described remote measurement stations.

The control system 10 of FIG. 5 thus receives local sensor data from local pressure sensors 80-S and 80-R as well as primary remote sensor data from the primary remote pressure sensors 90-1S and 90-1R and auxiliary remote sensor data from the auxiliary remote pressure sensors 90-2S and 90-2R. The control system 10 is configured to control operation of the pump 7 based on the thus received local sensor data, primary remote sensor data and auxiliary remote sensor data, e.g. as described in connection with FIGS. 6B and 8A-B.

In the following, embodiments of a control process for operating a pump of a fluid system will be described.

FIGS. 6A and 6B show state diagrams of two examples of a control process for operating a pump of a fluid system. The process may be performed by a control system for controlling operation of a pump as disclosed herein.

In particular, FIG. 6A shows a state diagram of an examples of a control process for operating a pump of a fluid system that, in addition to a local differential pressure sensor or a pair of local pressure sensors, includes a single remote differential pressure sensor or a single pair of remote pressure sensors, e.g. as illustrated in FIG. 1 or FIG. 4. The control process normally operates in an “online” state S0, where the process is able to receive remote sensor data from the remote pressure sensor(s). In the online state S0, the control system controls the pump based on the currently received local sensor data as well as on the currently received remote sensor data. An example of a control process performed while in the online state S0, will be described in greater detail below with reference to FIG. 7A.

When the control process detects a failure to receive the remote sensor data, the process changes into an ‘offline” state S-OFF, as indicated by arrow 601. It will be appreciated that, in some embodiments, the process may transition to the offline state S-OFF only when the failure to receive the remote sensor data continues for a minimum outage period, e.g. for at least several minutes, such as at least for 15 minutes or another suitable choice of minimum outage period. In the offline state S-OFF, the control system controls the pump based only on the currently received local sensor data and, optionally, additionally based on previously received local sensor data, which was received and recorded and/or analyzed while the process was still operating in the online state S0. An example of a control process performed while in the online state, will be described in greater detail below with reference to FIG. 7B.

When the process detects that it again receives remote sensor data, the process transitions back from the offline state S-OFF to the online state S0, as indicated by arrow 602, and resumes normal operation in the online state S0, based on the currently received local and remote sensor data.

FIG. 6B shows a state diagram of an example of a control process for operating a pump of a fluid system that, in addition to a local differential pressure sensor or a pair of local pressure sensors, includes a primary remote differential pressure sensor (or a primary pair of remote pressure sensors) and additionally an auxiliary remote differential pressure sensor (or a pair of auxiliary remote pressure sensors), e.g. as illustrated in FIG. 5.

The process of FIG. 6B is similar to the process of FIG. 6A, in that it includes an “online” state S0, where the process is able to receive remote sensor data from the primary remote pressure sensor(s). In the online state S0, the control system controls the pump based on the currently received local sensor data as well as on the currently received primary remote sensor data. An example of a control process performed while in the online state of this and other embodiments will be described in greater detail below with reference to FIG. 8A.

The process of FIG. 6B differs from the process of FIG. 6A in that the process of FIG. 6B includes more than one offline state. In particular, when the control process detects a failure to receive the primary remote sensor data, or at least a failure that persists for a minimum outage period, the process changes into an “auxiliary state S1, as indicated by arrow 601. In the auxiliary state S1, the control system still receives auxiliary remote sensor data from the auxiliary remote pressure sensor(s) but does no longer receive current primary remote sensor data, i.e. the auxiliary state may be regarded as a partial offline state. Accordingly, in the auxiliary state S1, the control process controls the pump based on the currently received local sensor data and the currently received auxiliary sensor data. Additionally, the control may further be based on previously received remote sensor data from the primary and/or auxiliary remote sensor(s), i.e. on remote sensor data which was received while the process was still operating in the online state S0. An example of a control process performed while in the auxiliary state S1 of this and other embodiments will be described in greater detail below with reference to FIG. 8B.

When the process, while operating in the auxiliary state S1, detects a failure to receive the auxiliary remote sensor data as well, or at least a failure that persists for a minimum outage period, the process changes into an “offline” state S-OFF, as indicated by arrow 603. In the offline state S-OFF, the control system no longer receives any current remote sensor data. Accordingly, in the offline state S-OFF, the control system controls the pump based only on the currently received local sensor data and, optionally, additionally based on previously received local sensor data, which was received while the process was still operating in the online state S0 and/or the auxiliary state S1. Accordingly operation in the offline state S-OFF of the present embodiment is similar to the operation in the offline state of the process of FIG. 6A, an example of which will be described in greater detail below with reference to FIG. 7B.

When, while operating in the offline state S-OFF, the process detects that it again receives auxiliary remote sensor data, the process returns from the offline state S-OFF to the auxiliary state S1, as indicated by arrow 604. When, while operating in the auxiliary state S1 or in the offline state S-OFF, the process detects that it again receives primary remote sensor data, the process returns to the online state S1, as indicated by arrows 602 and 605, respectively.

It will be appreciated that alternative embodiments may include additional or alternative offline states. For example, when a fluid system includes more than two remote differential pressure sensors, e.g. as the system of FIG. 2, the process may include an online state, an offline state and more than one auxiliary states, corresponding to the number of available remote pressure sensors. It will further be appreciated that, in embodiments with more than one remote differential pressure sensors, the choice of which sensor is used as a primary pressure sensor and which sensor(s) is/are used as an auxiliary pressure sensors, and in which prioritized order, may be predetermined and/static, or the control process may adaptively select the primary and auxiliary pressure sensor(s), e.g. as described below with reference to FIG. 8A.

FIG. 7A schematically illustrates a process for operating a pump of a fluid system based on a measured local differential pressure and a measured primary remote differential pressure, in particular when operating in an online state as described in connection with FIG. 6A. To this end, the fluid system includes a pump 7, which, in this examples, is operationally coupled to a supply line 5 of the fluid system. The fluid system further includes a local differential pressure sensor 80 which provides local sensor data indicative of a differential pressure between the supply line 5 and a return line 6 of the fluid system at or near the location of the pump 7. The fluid system further includes a primary remote differential pressure sensor 90, which provides primary remote sensor data indicative of a primary remote differential pressure between the supply line 5 and a return line 6 of the fluid system at a primary remote measurement location remote from the pump 7, e.g. as described in connection with any one of FIGS. 1 and 4.

The control process compares the currently measured local differential pressure as measured by local differential pressure sensor 80 with a local pressure set point 81 to determine a corresponding control error e. Based on the control error e, the process controls 11 the pump speed of pump 7 so as to reduce the magnitude (e.g. the absolute value) of the control error e. To this end, the process may implement a PI control scheme or another suitable control scheme.

The process further utilizes the received primary remote sensor data obtained from primary remote differential pressure sensor 90 to adapt the local pressure set point 81. In particular, the process may compare the currently measured primary remote sensor data with a primary remote pressure set point, which is indicative of a desired target value of the primary remote differential pressure at the primary remote measurement location where the primary remote differential pressure sensor is located. The primary remote pressure set point may be a predetermined value chosen sufficiently high to ensure a sufficiently high differential pressure at the recipients. When the currently measured primary remote sensor data deviates from the primary remote pressure set point, the system may adapt the local pressure set point 81 so as to reduce the magnitude of the determined deviation between the primary remote sensor data and the primary remote pressure set point. In particular, by adapting the local pressure set point 81, the process will control pump operation such that the measured local sensor data, measured by local differential pressure sensor 80, approaches the thus adapted local pressure set point 81. The control of the local differential pressure relative at the pump location to the thus adapted local pressure set point 81 will also affect the primary remote sensor data measured at the remote location of primary remote pressure sensor 90.

In some embodiments, the process may adapt the local pressure set point 81 by a predetermined percentage of the determined deviation between the primary remote sensor data and the primary remote pressure set point, thereby providing a gradual adaptation of the local pressure set point 81.

The adaptation of the local pressure set point 81 may be performed on a slower time scale than the control 11 of the local differential pressure. For example, the local pressure control 11 may, in some embodiments be performed on a time scale of seconds or even faster, while the adaptation of the local pressure set point may be performed on a time scale of minutes or even tens of minutes.

Accordingly, the process of FIG. 7A provides a control of the differential pressure at the primary remote measurement location which is closer to the recipients than the pump, thereby providing a reliable yet energy-efficient pressure control, as was explained in connection with FIG. 3.

In particular, the process of FIG. 7A allows the pressure at the primary remote measurement location to be controlled to tightly follow the primary remote pressure set point.

Now turning to FIG. 7B, as described above, there may be situations when the control process loses communication to the primary remote pressure sensor, or otherwise fails to receive the primary remote sensor data. If the failure to receive the primary remote sensor data continues for at least a minimum outage period, the process changes to an offline state, e.g. as described in connection with FIG. 6A, and FIG. 7B illustrates an example of a control process when operating in an offline state, in particular in the absence of any current remote sensor data.

In such a situation the process can no longer continuously adapt the local pressure set point 81 based on the actual, currently measured primary remote differential pressure at a primary remote measurement location. Therefore, the process of FIG. 7B uses a fallback strategy 12 for adapting the local pressure set point 81. For example, the process may adjust the local pressure set point 81 to a predetermined fallback set point. The predetermined fallback set-point may be chosen large enough to ensure a sufficiently high differential pressure at the recipients even during high-flow conditions.

The predetermined fallback set point may be manually selected, e.g. during an initial configuration of the system. Alternatively, the fallback set point may be adaptively selected during operation of the system in its normal online state. To this end, the process may monitor the local differential pressure, as measured by local differential pressure sensor 80, during normal operation over a suitably long time window, e.g. for 1 h, or 6 h or 24 h, and select the predetermined fallback set point corresponding to the maximum local differential pressure recorded during a time window preceding the failure to receive the remote sensor data. The predetermined fallback set point may be selected to be equal to the recorded maximum local differential pressure, optionally increased by a predetermined safety margin.

FIGS. 8A-B illustrate an example of the pump operation in a fluid system with more than one remote pressure sensors, e.g. in the system of FIG. 2 or FIG. 5.

In particular, FIGS. 8A-B illustrate a process for operating a pump in a fluid system with three remote pressure sensors as in FIG. 1. However, it will be appreciated that the process may be performed in an analogous manner in case of a fluid system having two or more than three remote pressure sensors.

FIG. 8A illustrates the control process when the control system operates in its online state, e.g. as described in connection the online state S0 of FIG. 6B, while FIG. 8B illustrates the control process when the control system operates in a partial offline state, e.g. as described in connection the auxiliary state S1 of FIG. 6B.

The process of FIG. 8A is similar to the process of FIG. 7A in that it controls the pump speed based on an error signal e between a measured local differential pressure and an adaptive local pressure set point 81, which is adapted based on a deviation between primary remote sensor data, measured remotely from the pump, and a corresponding primary remote pressure set point. The process of FIG. 8A differs from the process of FIG. 7A in that the process receives remote sensor data from multiple remote pressure sensors 90-1 through 90-3. Therefore, the process includes a selection step 13 in which the process selects one of the multiple remote pressure sensors as a primary remote pressure sensor and the remaining remote pressure sensors as auxiliary remote pressure sensors in a prioritized order.

The selection/prioritization 13 may be performed in a variety of ways. In one embodiment, the process assigns remote pressure set points to the respective remote pressure sensors. For example, the remote pressure set points may be set, e.g. manually, at the time of configuration of the system. The remote pressure set points indicate the remote differential pressure to be maintained at the respective remote measurement locations where the remote pressure sensors are located. The remote pressure set points may all be set to the same value—e.g. when all remote measurement locations are located at the edge of the grid- or they may have respective predetermined values. The process may monitor operation of the fluid system, when in online mode, and compare the remote sensor data from the remote pressure sensors 90-1 through 90-3 with their respective remote pressure set points and sort the remote pressure sensors based on the average or maximum deviation of the respective measured remote sensor data from their respective remote pressure set points. The process may then select the remote pressure sensor having the smallest deviation as the primary remote pressure sensor and create a prioritized list of auxiliary remote pressure sensors by sorting the remaining remote pressure sensors according to increasing deviation from their respective remote pressure set points, the remote pressure sensors having the smallest deviation among the remaining remote pressure sensors being selected as the highest-ranking auxiliary remote pressure sensor. It will be appreciated that the process may repeat the selection of the remote pressure sensor having the smallest deviation as the primary remote pressure sensor and the creation of the prioritized list of auxiliary remote pressure sensors, e.g. periodically or otherwise repeatedly. Accordingly, the remote measurement location based on which the adaptive local pressure set point is adapted may vary over time, e.g. depending on the varying load distribution within the fluid system.

In the example of FIG. 8A, it is assumed that the process has selected remote pressure sensor 90-1 as the primary remote pressure sensor, remote pressure sensor 90-2 as the highest-ranking auxiliary remote pressure sensor and remote pressure sensor 90-3 as the next-highest-ranking remote pressure sensor.

During normal operation in its online state, i.e. when current sensor data from the thus selected primary pressure sensor is available, the process adjusts the local pressure set point 81 based on the primary remote sensor data currently obtained from the selected primary remote pressure sensor 90-1, e.g. in the manner described above in connection with FIG. 7A, i.e. by adapting the local pressure point 81 by a predetermined percentage of the deviation between the primary remote sensor data and its associated primary remote pressure set point. Even though the auxiliary pressure sensors 90-2 and 90-3 are not directly used for the pump control when the control process operates in its online state, the auxiliary remote sensor data received from the auxiliary remote pressure sensors is still recorded and/or analyzed while the process operates in the online state, so the data is available for potential use in case of a failure to receive the primary remote sensor data.

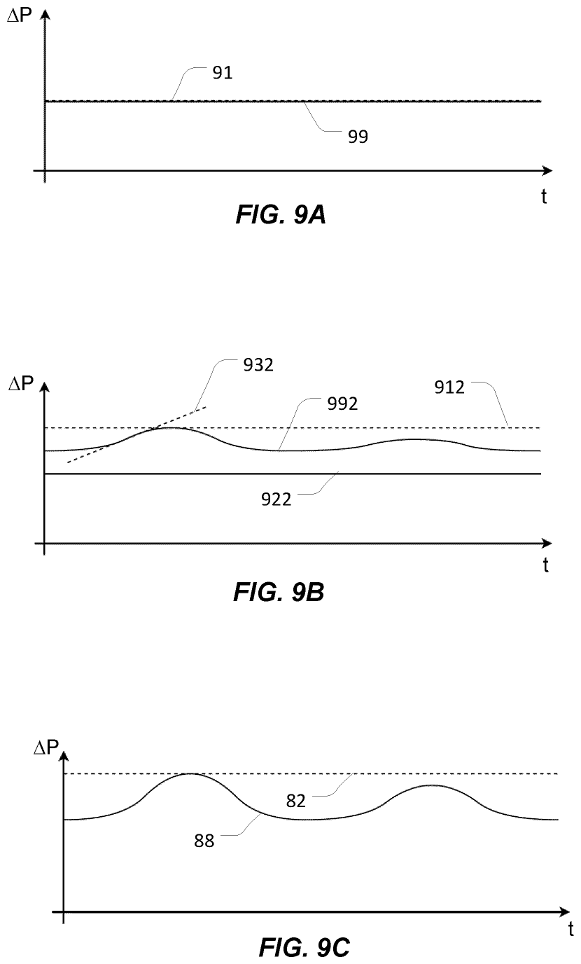

This is schematically illustrated in FIGS. 9A-C, which illustrate the time-varying primary remote sensor data, the highest-ranking auxiliary remote sensor data and the local sensor data, respectively.

In particular, FIG. 9A illustrates the predetermined remote pressure set point (dotted line 81) and the actually measured primary remote sensor data (solid line 88) as a function of time, when the control process of FIG. 8A has reached a steady state.

FIG. 9B illustrates an example of the time-varying auxiliary remote sensor data 992 from the highest-ranking auxiliary remote pressure sensor 90-2 as monitored during operation in the online state. As will be appreciated from FIG. 9B, the auxiliary remote sensor data 992 will typically vary over time in response to changes in the flow and/or changes in the pump speed. In particular, the auxiliary remote sensor data 992 may often be higher than the corresponding auxiliary remote set point 922, since the pump control is based on the primary remote sensor data, which had been selected to be the remote sensor data having the smallest deviation relative to its primary remote pressure set point. As will further be appreciated from FIG. 9B, the control process may monitor the auxiliary remote sensor data 992 so as to determine a maximum value 912 over a predetermined time window and, optionally, a maximum rate of change 932 during said time window. The determination of the maximum value 912 and maximum rate of change 932 may be determined using standard techniques for evaluating time series. In particular, the process may perform a low-pass filtering of the received sensor data, so as to avoid that the maximum values are influenced by measurement noise or short peaks in the measured values.

FIG. 9C shows the corresponding local differential pressure 88 at the location of the pump. The local differential pressure varies over time, responsive to the operation of the pump, which is controlled according to a time-varying local pressure set point. During operation in the online state, the time-varying local pressure set point is continuously or intermittently adapted so as to maintain a constant remote differential pressure 99 as illustrated in FIG. 9A, thus resulting in a time-varying local differential pressure.

Turning to FIG. 8B, as described above, there may be situations when the control process loses communication to the primary remote pressure sensor 90-1, or otherwise fails to receive the primary remote sensor data. If the failure to receive the remote sensor data continues for at least a minimum outage period, and if the process still receives remote sensor data from one or more of the auxiliary remote pressure sensors, the process changes to a partial offline state, e.g. as described in connection with the auxiliary state S1 of FIG. 6B.

FIG. 8B illustrates an example of a control process when operated in a partial offline state, in particular in the absence of the primary remote sensor data but with continued receipt of the highest-ranking auxiliary remote sensor data.

In such a situation the process can no longer continuously adapt the local pressure set point based on the actual, currently measured primary remote sensor data. Therefore, the process of FIG. 8B uses a fallback strategy 14 for adapting the local pressure set point 81 based on the highest-ranking auxiliary remote sensor data that is still being received. In the example of FIG. 8B, this is assumed to be the remote sensor data from remote pressure sensor 90-2.

In particular, the fallback strategy 14 adapts the local pressure set point 81 based on a maximum differential pressure measured by the highest-ranking auxiliary remote pressure sensor 90-2 during a time window preceding the detected failure, such as during a time window immediately preceding the detected failure. To this end, the process may analyze recorded auxiliary remote sensor data, recorded during a time window preceding the detected failure, such as a time window having a predetermined duration of most recent available auxiliary remote sensor data preceding the detected failure. As was illustrated in FIG. 9B, the process may determine a maximum value of the highest-ranking auxiliary remote sensor data during the selected time window and, optionally, a maximum rate of change 932 of the highest-ranking auxiliary remote sensor data during the same or a different time window. The duration of the time window(s) may be selected sufficiently long to capture typical pressure variations over a time period corresponding to an expected duration of the failure. The choice of time window may depend of the geographic region and, in particular, the typical climate variations at the location of the fluid system. Other factors that may influence the choice of time window may include the type of fluid system. Examples of suitable durations may range between 10 min, and 1 month, such as between 10 min, and 1 week, such as between 30 min, and 48 h, such as between 30 min, and 24 h, e.g. 1 h or 6 h or 24 h. In some embodiments, the process initially selects a relative short time window, e.g. 1 h. If the detected failure continuous for a longer period, e.g. longer than the selected duration of the time window, the process may select a new, longer time window, e.g. a 6 h time window, at determine a new maximum value 912 over the new, longer time window. The process may then use the newly computed maximum value during the continued control of the pump. This may be repeated if necessary, e.g. if the failure continues for long time periods, until a predetermined maximum time window is reached. It will further be appreciated that the process may select the same or different time windows for the determination of the maximum value 912 and the maximum rate of change 932, respectively.

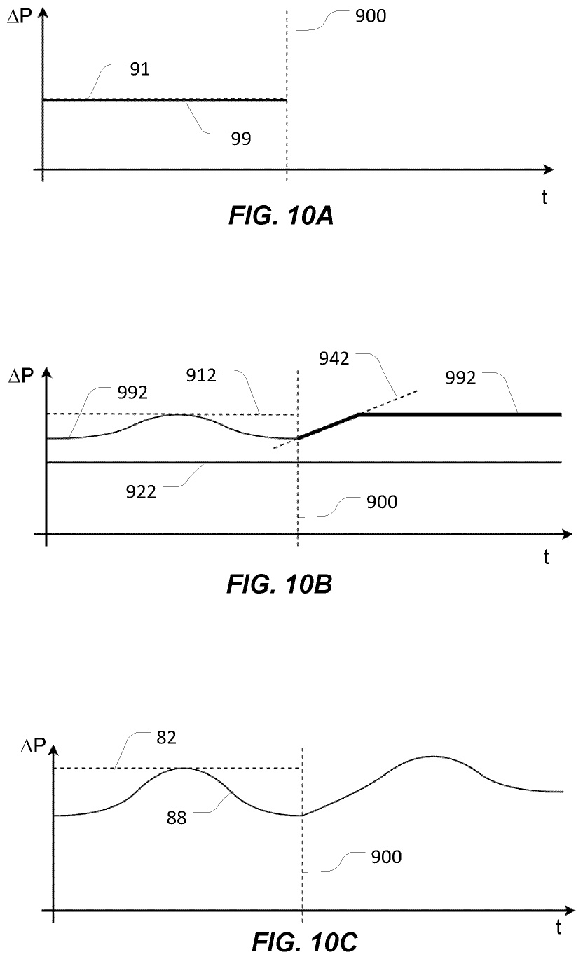

FIGS. 10A-10C illustrate an example of the pump control in a situation where a failure to receive the primary remote sensor data occurs. In particular, FIGS. 10A-C schematically illustrate the received measured primary remote sensor data, the highest-ranking auxiliary remote sensor data, and the local sensor data, respectively, before and after a detected failure to receive the primary remote sensor data. In the illustrated example, a failure situation is illustrated where, from a time 900 onwards, the process no longer receives the primary remote sensor data.

In particular, FIG. 10A illustrates the primary remote sensor data 99 and its associated primary remote pressure set point 91. FIG. 10B illustrates the highest-ranking auxiliary remote sensor data 992 and its recorded maximum value 912 during a predetermined period preceding the time of failure 900. Finally, FIG. 10C illustrates the local sensor data 88 and its maximum value 82 as detected during a time window prior to the time 900 of failure to receive the primary remote sensor data.

From the time 900 of the detected failure onwards, the process no longer receives the primary remote sensor data 99. Instead, the fallback strategy determines the maximum value 912 of the highest-ranking auxiliary remote sensor data 992 during a time window prior to the time 900 of failure, as well as the maximum rate of change (designated 932 in FIG. 9B) during said time window. The process then gradually adapts, in particular increases, the local pressure set point 81 until the currently received highest-ranking auxiliary remote sensor data corresponds to the computed maximum value 912. The process may control the rate 942 of the gradual increase to correspond to the computed maximum rate of change 932 of the highest-ranking auxiliary remote sensor data preceding the detected failure. This fallback strategy ensures that, in the absence of the primary remote sensor data, the local pressure 88 is controlled such that the highest-ranking auxiliary pressure is increased to a value that corresponds to the actually observed maximum during a period where the control was still based on the primary remote sensor data. Moreover, the rate of the increase is limited to the previously recorded maximum rate of change, thus avoiding an unnecessarily abrupt increase of the pressure.

As is illustrated in FIG. 10C, the increase of the local pressure set point 81 causes the local pressure control loop 11 to gradually increase the local differential pressure 88 and to continue the local pressure control based on the adapted local pressure set point 81. As mentioned above, in some embodiments, if the failure situation continues over an extended period of time, in particular for longer than the duration of the time window used to determine the maximum value 912, the process may compute a new maximum value 912 based on a longer time window, and further adapt the local pressure set point 81 based on that new maximum value. This may be repeated until a maximum duration is reached, which may be 24 h or 48 h or another maximum duration.

If the system also fails to receive the highest-ranking auxiliary remote sensor data, the process may proceed with the next-highest-ranking auxiliary remote sensor data, i.e. by determining a maximum value of the next-highest-ranking auxiliary remote sensor data preceding the failure to receive the primary remote sensor data, and by adjusting the local pressure set point based in the thus computed maximum value.

When the failure situation ends and the process again receives primary remote sensor data, the process may resume normal operation based on the normal control strategy, e.g. as illustrated in FIG. 10A. This will typically involve a decrease of the local pressure set point 81. Preferably the decrease is performed gradually, as described above.

It will be appreciated that a number of modifications may be made to the process and system described herein.

For example, the process may define a minimum and maximum pressure and limit the pressure control to be within a pressure range between the minimum and maximum pressure. Additionally or alternatively, the process may adjust the remote sensor data, e.g. based on known differences in elevation of the respective measurement locations.

It will further be appreciated that the pump may be operationally coupled to a return line of the return grid instead of to a supply line. Accordingly, according to one aspect, disclosed herein are embodiments of a method of controlling operation of a pump to control a fluid pressure in a fluid system, such as a fluid-based energy distribution system. In particular, the fluid system comprises a supply grid of supply lines for transporting fluid from a distribution source to respective recipients, a return grid of return lines for returning fluid from the respective recipients to the distribution source, and a pump for pumping fluid through the fluid system, in particular through a supply line of the supply grid or through a return line of the return grid. Various embodiments of the method comprise:
receiving local sensor data indicative of a local differential pressure between a local supply line measurement location along a supply line of the supply grid and a local return line measurement location along a return line of the return grid; receiving primary remote sensor data indicative of a primary remote differential pressure between a primary remote supply line measurement location along a supply line of the supply grid and a primary remote return line measurement location along a return line of the return grid, wherein the primary remote supply line measurement location is further displaced along the supply line downstream from the pump than the local supply line measurement location and/or wherein the primary remote return line measurement location is further displaced along the return line from the pump, in particular upstream from the pump, than the local return line measurement location;
controlling operation of the pump based on at least the received local sensor data, the received primary remote sensor data and on a local pressure set point;
responding to a detected failure to receive the primary remote sensor data at least by modifying, in particular increasing, the local pressure set point and by controlling operation of the pump based on at least the received local sensor data and the modified local pressure set point.

While the various aspects disclosed herein have mainly been described in the context of a district heating system, it will be appreciated that they may also be applied to other types of fluid systems.

Some embodiments of the various aspects disclosed herein may be summarized as follows:
Embodiment 1: A method of controlling operation of a pump to control a fluid pressure in a fluid system, in particular in a fluid-based energy distribution system, the fluid system comprising a supply grid of supply lines for transporting fluid from a distribution source to respective recipients, a return grid of return lines for returning fluid from the respective recipients to the distribution source, and a pump for pumping fluid through a supply line of the supply grid, wherein the method comprises:
   receiving local sensor data indicative of a local differential pressure between a local supply line measurement location along a supply line of the supply grid and a local return line measurement location along a return line of the return grid;
   receiving primary remote sensor data indicative of a remote differential pressure between a primary remote supply line measurement location along a supply line of the supply grid and a primary remote return line measurement location along a return line of the return grid, wherein the primary remote supply line measurement location is further displaced along the supply line downstream from the pump than the local supply line measurement location;
   controlling operation of the pump based on at least the received local sensor data, the received primary remote sensor data and on a local pressure set point;
   responding to a detected failure to receive the primary remote sensor data at least by modifying, in particular increasing, the local pressure set point and by controlling operation of the pump based on at least the received local sensor data and the modified local pressure set point.
Embodiment 2: The method according to embodiment 1, wherein the local pressure set point is indicative of a target differential pressure between the local supply line measurement location and the local return line measurement location.
Embodiment 3: The method according to embodiment 1 or 2, wherein modifying the local pressure set point and controlling operation of the pump based on at least the received local sensor data and the modified local pressure set point comprises:
   incrementally modifying, in particular incrementally increasing, the local pressure set point, and,
   after each incremental modification of the local pressure set point, controlling operation of the pump based on at least the received local sensor data and the incrementally modified local pressure set point.
Embodiment 4: The method according to any one of the preceding embodiments, comprising:
   responding to a detected resumption of receipt of the primary remote sensor data at least by further modifying, in particular decreasing, the modified local pressure set point and by controlling operation of the pump based on at least the received local sensor data, the received primary remote sensor data and the further modified local pressure set point.
Embodiment 5: The method according to any one of the preceding embodiments, wherein controlling operation of the pump based on at least the received local sensor data, the received primary remote sensor data and the local pressure set point comprises:
   computing a deviation of the received primary remote sensor data from a primary remote pressure set point;
   adapting the local pressure set point based on the computed deviation, in particular so as to reduce a magnitude of the computed deviation.
Embodiment 6: The method according to any one of the preceding embodiments, further comprising:
   receiving auxiliary remote sensor data indicative of an auxiliary remote differential pressure between an auxiliary remote supply line measurement location along a supply line of the supply grid and an auxiliary remote return line measurement location along a return line of the return grid,
wherein modifying the local pressure set point and controlling operation of the pump based on at least the received local sensor data and the modified local pressure set point comprises:
   modifying the local pressure set point based on the auxiliary remote sensor data, received prior to the detected failure, and controlling operation of the pump based on at least the received local sensor data, the currently received auxiliary remote sensor data and on the modified local pressure set point.
Embodiment 7: The method according to embodiment 6, wherein modifying the local pressure set point based on the auxiliary remote sensor data comprises:
   computing a maximum auxiliary remote differential pressure observed during a time window preceding the detected failure, and
   modifying the local pressure set point based on a deviation of the currently observed auxiliary remote sensor data from the computed maximum auxiliary remote differential pressure.
Embodiment 8: The method according to embodiment 7, wherein modifying the local pressure set point based on the auxiliary remote sensor data comprises:
   computing a maximum rate of change of the auxiliary remote differential pressure observed during a time window preceding the detected failure, and
   gradually modifying the local pressure set point at a rate corresponding to the computed maximum rate of change.
Embodiment 9: The method according to embodiment 7 or 8, comprising selecting a length of the time window responsive to a detected duration of the failure to receive the primary remote sensor data.
Embodiment 10: The method according to any one of embodiments 6 through 9, further comprising:
   receiving remote sensor data indicative of respective remote differential pressures at respective sets of remote measurement locations, each set of remote measurement locations having a respective remote pressure set point associated with it,
   selecting remote sensor data associated with a first one of the respective sets of respective remote measurement locations as the primary remote sensor data, and
   selecting sensor data from associated with a second one of the respective sets of respective remote measurement locations as the auxiliary remote sensor data.
Embodiment 11: The method according to embodiment 10, wherein selecting comprises:
   for each of the sets of remote measurement locations, determining a deviation between the remote sensor data measured at said set of remote measurement locations and the remote pressure set point associated with said set of remote measurement locations, and
   selecting the remote sensor data of the set of remote measurement locations having the smallest deviation as the primary remote sensor data.
Embodiment 12: The method according to embodiment 11, further comprising selecting the remote sensor data from the set of remote measurement locations having the second to smallest deviation as the auxiliary remote sensor data.
Embodiment 13: The method according to any one of embodiments 10 through 12, wherein the sets of remote measurement locations are located at respective peripheral portions of the supply and return grids.
Embodiment 14: The method according to any one of the preceding embodiments, wherein detecting a failure to receive the primary remote sensor data comprises detecting a failure to receive the primary remote sensor data for at least a predetermined minimum outage period.
Embodiment 15: A control system for controlling operation of a pump to control a fluid pressure in a district energy system, the control system being configured to perform the steps of the method according to any one of the preceding embodiments.
Embodiment 16: A fluid system, comprising:
   a distribution source for providing a fluid,
   a supply grid of supply lines for feeding the fluid from the distribution source to a plurality of recipients,
   a return grid of return lines for returning fluid from the plurality of recipients to the distribution source,
   a pump for pumping fluid through a supply line of the supply grid;
   a control system as described herein for controlling the pump,
   at least one local pressure sensor communicatively coupled to the control system and configured for providing local sensor data indicative of a local differential pressure between a local supply line measurement location along a supply line of the supply grid and a local return line measurement location along a return line of the return grid,
   at least one primary remote pressure sensor communicatively coupled to the control system and configured for providing primary remote sensor data indicative of a primary remote differential pressure between a primary remote supply line measurement location along a supply line of the supply grid and a primary remote return line measurement location along a return line of the return grid, wherein the primary remote supply line measurement location is further displaced along the supply line downstream from the pump than the local supply line measurement location.
Embodiment 17: A computer program comprising program code configured to cause, when executed by a data processing system, the data processing system to perform the steps of the method according to any one of embodiments 1 through 14.

Various embodiments of Embodiments of the method described herein may be computer-implemented. In particular, embodiments of the method may be implemented by means of hardware comprising several distinct elements, and/or at least in part by means of a suitably programmed data processing system. In the apparatus claims enumerating several means, several of these means can be embodied by one and the same element, component or item of hardware. The mere fact that certain measures are recited in mutually different dependent claims or described in different embodiments does not indicate that a combination of these measures cannot be used to advantage.

It should be emphasized that the term “comprises/comprising” when used in this specification is taken to specify the presence of stated features, elements, steps or components but does not preclude the presence or addition of one or more other features, elements, steps, components or groups thereof.

1. A method of controlling operation of a pump to control a fluid pressure in a fluid system, in particular in a fluid-based energy distribution system, the fluid system comprising a supply grid of supply lines for transporting fluid from at least one distribution source to respective recipients, a return grid of return lines for returning fluid from the respective recipients to the distribution source, and a pump for pumping fluid through a supply line of the supply grid, wherein the method comprises the steps of:
receiving local sensor data indicative of a local differential pressure between a local supply line measurement location along a supply line of the supply grid and a local return line measurement location along a return line of the return grid;
receiving primary remote sensor data indicative of a remote differential pressure between a primary remote supply line measurement location along a supply line of the supply grid and a primary remote return line measurement location along a return line of the return grid, wherein the primary remote supply line measurement location is further displaced along the supply line downstream from the pump than the local supply line measurement location;
controlling operation of the pump based on at least the received local sensor data, the received primary remote sensor data and on a local pressure set point;
responding to a detected failure to receive the primary remote sensor data at least by modifying the local pressure set point and by controlling operation of the pump based on at least the received local sensor data and the modified local pressure set point.

2. The method according to claim 1, wherein the local pressure set point is indicative of a target differential pressure between the local supply line measurement location and the local return line measurement location.

3. The method according to claim 1, wherein modifying the local pressure set point and controlling operation of the pump based on at least the received local sensor data and the modified local pressure set point comprises:
incrementally modifying the local pressure set point, and,
after each incremental modification of the local pressure set point, controlling operation of the pump based on at least the received local sensor data and the incrementally modified local pressure set point.

4. The method according to claim 1, comprising:
responding to a detected resumption of receipt of the primary remote sensor data at least by further modifying the modified local pressure set point and by controlling operation of the pump based on at least the received local sensor data, the received primary remote sensor data and the further modified local pressure set point.

5. The method according to claim 1, wherein controlling operation of the pump based on at least the received local sensor data, the received primary remote sensor data and the local pressure set point comprises:
computing a deviation of the received primary remote sensor data from a primary remote pressure set point;
adapting the local pressure set point based on the computed deviation, in particular so as to reduce a magnitude of the computed deviation.

6. The method according to claim 1, further comprising:
receiving auxiliary remote sensor data indicative of an auxiliary remote differential pressure between an auxiliary remote supply line measurement location along a supply line of the supply grid and an auxiliary remote return line measurement location along a return line of the return grid,
wherein modifying the local pressure set point and controlling operation of the pump based on at least the received local sensor data and the modified local pressure set point comprises:
modifying the local pressure set point based on the auxiliary remote sensor data, received prior to the detected failure, and controlling operation of the pump based on at least the received local sensor data, the currently received auxiliary remote sensor data and on the modified local pressure set point.

7. The method according to claim 6, wherein modifying the local pressure set point based on the auxiliary remote sensor data comprises:
computing a maximum auxiliary remote differential pressure observed during a time window preceding the detected failure, and
modifying the local pressure set point based on a deviation of the currently observed auxiliary remote sensor data from the computed maximum auxiliary remote differential pressure.

8. The method according to claim 7, wherein modifying the local pressure set point based on the auxiliary remote sensor data comprises:
computing a maximum rate of change of the auxiliary remote differential pressure observed during a time window preceding the detected failure, and
gradually modifying the local pressure set point at a rate corresponding to the computed maximum rate of change.

9. The method according to claim 7, comprising selecting a length of the time window responsive to a detected duration of the failure to receive the primary remote sensor data.

10. The method according to claim 6, further comprising the steps of:
receiving remote sensor data indicative of respective remote differential pressures at respective sets of remote measurement locations, each set of remote measurement locations having a respective remote pressure set point associated with it,
selecting remote sensor data associated with a first one of the respective sets of respective remote measurement locations as the primary remote sensor data, and
selecting sensor data from associated with a second one of the respective sets of respective remote measurement locations as the auxiliary remote sensor data.

11. The method according to claim 10, wherein selecting comprises:
for each of the sets of remote measurement locations, determining a deviation between the remote sensor data measured at said set of remote measurement locations and the remote pressure set point associated with said set of remote measurement locations, and
selecting the remote sensor data of the set of remote measurement locations having the smallest deviation as the primary remote sensor data.

12. The method according to claim 11, further comprising selecting the remote sensor data from the set of remote measurement locations having the second to smallest deviation as the auxiliary remote sensor data.

13. The method according to claim 10, wherein the sets of remote measurement locations are located at respective peripheral portions of the supply and return grids.

14. The method according to claim 1, wherein detecting a failure to receive the primary remote sensor data comprises detecting a failure to receive the primary remote sensor data for at least a predetermined minimum outage period.

15. A control system for controlling operation of a pump to control a fluid pressure in a district energy system, the control system being configured to perform the steps of the method according to claim 1.

16. A fluid system, comprising:
at least one distribution source for providing a fluid,
a supply grid of supply lines for feeding the fluid from the distribution source to a plurality of recipients,
a return grid of return lines for returning fluid from the plurality of recipients to the distribution source,
a pump for pumping fluid through a supply line of the supply grid;
a control system as described herein for controlling the pump,
at least one local pressure sensor communicatively coupled to the control system and configured for providing local sensor data indicative of a local differential pressure between a local supply line measurement location along a supply line of the supply grid and a local return line measurement location along a return line of the return grid,
at least one primary remote pressure sensor communicatively coupled to the control system and configured for providing primary remote sensor data indicative of a primary remote differential pressure between a primary remote supply line measurement location along a supply line of the supply grid and a primary remote return line measurement location along a return line of the return grid, wherein the primary remote supply line measurement location is further displaced along the supply line downstream from the pump than the local supply line measurement location.

17. A computer program comprising program code configured to cause, when executed by a data processing system, the data processing system to perform the steps of the method according to claim 1.
